(19) United States
(12) Patent Application Publication
(10) Pub. No.: US 2007/0209939 A1
(43) Pub. Date: Sep. 13, 2007

(54) Two-dimensional transfer device

(75) Inventors: Stephen Haralampu, Belmont, MA (US); William M. Skea, Saco, ME (US); Mark Chlenov, Brighton, MA (US); Edward Thompson, Hudson, NH (US)

Correspondence Address: PATREA L. PABST;PABST PATENT GROUP LLP, 400 COLONY SQUARE, SUITE 1200, 1201 PEACHTREE STREET, ATLANTA, GA, 30361, US

(73) Assignee: Protein Forest, Inc.

(21) Appl. No.: 11/372,718

(22) Filed: Mar. 10, 2006

(51) Int. Cl. C07K 1/26; G01N 27/00

(52) U.S. Cl. 204/456; 204/606

(57) ABSTRACT

A system including apparatus and method of use that insures uniform contact and transfer of analytes from a first dimensional electrophoretic analytical gel to a second dimension, such as an electrophoretic analytical gel, has been developed. The device provides a means for transfer of analytes from a first dimension isoelectric focusing gel to a second dimension electrophoretic analytical gel. In a preferred embodiment, the device facilitates the transfer of protein analytes from a digital proteome chip to a second dimension electrophoretic analytical gel. The second dimension electrophoretic analytical gel can be for use with any technique known in the art, such as, but not limited to SDS-PAGE molecular weight analysis, secondary isoelectric focusing, and capillary electrophoresis. The main features of the system are a mechanical device that locates the first dimension analysis at a fixed and known position in space, and a low density medium that assures uniform electrical contact between the first and second dimension analyses. The advantage of an apparatus that places the first dimension in a known position is that the final data analysis is greatly facilitated by precisely knowing the locations of the first dimension pH features. The medium that assures electrical contact also has the advantage of simplifying the final data analysis by assuring a uniform frame of reference on how the second dimension analysis was run.

### BACKGROUND OF THE INVENTION

The present invention is generally in the field of devices to facilitate uniform transfer of sample from an electrophoretic device to a gel for electropheresis in a second dimension.

Methods and devices for performing two dimensional electrophoretic analyses are known in the art, especially for protein analyses, and are effective for separating and quantifying extremely complex mixtures. In its most common embodiment, a first dimension separation of proteins according to their isoelectric point, pI (the pH at which an analyte has zero mobility in an electric field), is followed by a second dimension molecular weight separation in a direction orthogonal to the first. The resulting two-dimensional image effectively resolves complex mixtures and identifies specific analytes according to their pI and molecular weight. Since it is typical that the isoelectric focusing is accomplished on a medium with a continuously changing pH gradient, it is critical to know the position where the first dimension is interfaced with the second dimension so that during the final data analysis an accurate estimate of the pI can be made. During the second dimension analysis it is important that unnecessary curvature, which could interfere with the final data interpretation, is not introduced. Commonly, curvature in second dimension analyses is a result of non-uniformity of the electric field.

It is an object of the present invention to provide a device and method for use in transferring separated sample from a first dimensional analytical gel to a second dimensional analytical gel.

It is a further object of the present invention to provide a device and method which minimizes disortion of the sample analysis, as well as labor and technical difficulty of the transfer.

### BRIEF DESCRIPTION OF INVENTION

A system including apparatus and method of use that insures uniform contact and transfer of analytes from a first dimensional electrophoretic analytical gel to a second dimension, such as an electrophoretic analytical gel has been developed. The device provides a means for transfer of analytes from a first dimension isoelectric focusing gel to a second dimension electrophoretic analytical gel. In a preferred embodiment, the device facilitates the transfer of protein analytes from a digital proteome chip to a second dimension electrophoretic analytical gel. The second dimension electrophoretic analytical gel can be for use with any technique known in the art, such as, but not limited to SDS-PAGE molecular weight analysis, secondary isoelectric focusing, and capillary electrophoresis.

The main features of the system are a mechanical device that locates the first dimension analysis at a fixed and known position in space, and a low density medium that assures uniform electrical contact between the first and second dimension analyses. The advantage of an apparatus that places the first dimension in a known position is that the final data analysis is greatly facilitated by precisely knowing the locations of the first dimension pH features. The medium that assures electrical contact also has the advantage of simplifying the final data analysis by assuring a uniform frame of reference on how the second dimension analysis was run. The electric field is directed through the gel plug, which can result in electrical leakage around the gel plug, which yields poor efficiency. The collar device fits well into automation, in contrast to gel strips.

### BRIEF DESCRIPTION OF THE DRAWINGS

FIG. 1 depicts a transfer device to a vertically run slab gel.

FIG. 2 depicts a horizontal transfer device format.

### DETAILED DESCRIPTION OF THE INVENTION

First dimension, pI-based separations are a common practice in the analysis of complex protein mixtures. To accomplish this, in general, soluble proteins are forced to migrate in an electric field in the presence of a pH gradient. The protein analytes attain an apparent positive charge at pH values below their pI, and will migrate toward the cathode, while the opposite is true at pH values above their pI. The pH gradient is arranged such that the lowest pH values are toward the anode end of the device, and the highest pH values are toward the cathode. Proteins stop migrating when they reach the pH where their electrophoretic mobility reaches zero, i.e., their pI. Proteins can be analyzed in either their native or denatured states, using substances like urea or thiourea or other commonly used denaturants. The pH gradients are commonly established via the pH ordering of a mixture of amphoteric buffers, known in the art as carrier ampholytes, in an electric field, or by the copolymerization of a gradient of acid and base moieties within the structure of a polyacrylamide gel, known as immobilized pH gradients (IPG).

Alternatively, U.S. published patent application 20030102215A1 to Zilberstein and Bukshpan disclose a discrete pH trapping device, referred to as the digital proteome chip, or dPC. In the dPC, an array containing a multiplicity of discrete pH features serves as a permeable partition between an acidic anode buffer chamber and a basic cathode buffer chamber. Proteins below their pI in the anode chamber exhibit a net positive charge and migrate toward the cathode through the pH features that maintain the protein below its pI. Conversely, proteins above their pI in the cathode chamber exhibit a net negative charge, and migrate toward the anode through the pH features that maintain the protein above its pI. Proteins tend to accumulate in the pH features closest to their pI, where their net motion is either zero at the pI, or very slow near it. The advantage of the dPC is that by its discrete nature the pH of any specific feature is known according to its formulation, rather than by being extrapolated from known endpoints, as is done in the carrier ampholyte or IPG systems. A characteristic of the dPC system is that the electrophoretic migration of the analytes is not parallel to the pH gradient, but random.

Complex mixtures can be further separated. A very common practice after isoelectric separations is to further separate the analytes according to their molecular weight. Many techniques are utilized in the art to accomplish this. As an illustrative example, the gel device from the first dimension is equilibrated with an ionic surfactant, such as sodium dodecylsulfate (SDS), to impart a uniform charge density to the analytes. These analyte-surfactant complexes are separated according to their molecular weight by observing their electrophoretic migration through a restrictive slab gel. It is critical in the accurate evaluation of electrophoretic migration that the rate of transfer of analytes from the first dimension separation to the second dimension be uniform.

A device that holds the first dimension at a known position relative to the second dimension is critical to insure intimate contact with the second dimension gel and to provide a lateral reference for the boundary positions of the first dimension. It is usual in conventional isoelectric focusing for the transfer to be to a slab polyacrylamide gel. In the case of the dPC device, the second dimension can be a slab, if the pH features are arranged in a linear array, or alternatively it can be a multiplicity of columns arranged in a pattern that assures intimate contact with each pH feature of the dPC. The advantage of the dPC arrangement is that features of known pH are held in one-to-one correspondence with locations on the second dimension analysis.

In the most common execution of a two dimensional electrophoretic analysis, the second dimension consists of a molecular weight based separation. To accomplish this, analytes separated in the first dimension are complexed with a surfactant, such as sodium dodecylsulfate, that imparts a uniform particle charge density. The protein analyte-surfactant complexes are formed by passive diffusion, or by electrophoretic movement of the surfactant into the first dimension analytical gel. It is advantageous to have an extended stacking gel region that mitigates any inconsistencies in the transfer rate of protein analytes. Any stacking gel, as is known in the art, can be used for this purpose, such as, but not limited to, a low percentage polyacrylamide (less than about 6%) or agarose (less than about 3%). The stacking gel must be greater than 0.5 mm thick and is preferably between 1 and 30 mm.

Other types of devices may be used in the second dimension, including capillary electropheresis, liquid chromatography, or direct mass spectroscopy device where the first dimension is a matrix and the second dimension or mass spectroscopy is positioned so that the plugs all line up.

To further assure uniformity of contact and analyte transfer between the first and second dimensions, it is advantageous to provide a conductive fluid medium that is non-restrictive to analyte flow, and that serves to fill any gaps between the first and second dimensions. Second dimension running buffers are known in the art can be used, but these have the disadvantage of occasionally flowing out of the critical contact region. In one embodiment, the stacking gel is cast in place and in contact between the first and second dimensions.

Alternatively, a flowable gel, such as, but not limited to, linear polyacrylamide, methyl cellulose, hydroxypropyl methyl cellulose, ethyl cellulose, cellulose ether, xanthan, uncharged polysaccharides, or polyols, or mixtures thereof, can be utilized. The gel must have a low enough apparent viscosity for easy application, but a high enough viscosity so that the gel does not flow out of place within the timescale of the second dimension analysis.

Any of the contact media used between the first and second dimensions may also contain additive components that assist in the electrophoretic migration of the analytes, such as buffers, and/or dyes, such as bromophenol blue, that aid in the visualization of the electrophoresis progress.

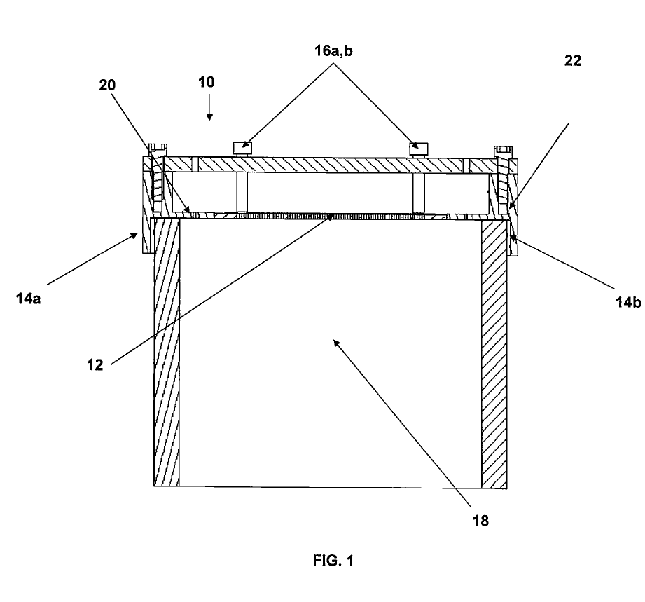

FIG. 1 depicts a transfer device 10 to a vertically run slab gel. The transfer device has the capability of uniquely locating a first dimension analytical gel, such as the dPC, and a mechanism by which the first dimension is firmly held in place. In FIG. 1, the dPC 12 is located by placing it in a recessed location 14a.b on the transfer collar 10. The dPC 12 is prevented from inadvertent movement by retaining screws or clamps 16a,b. In a typical utilization of the device depicted in FIG. 1, a second dimension cassette 18 containing a molecular weight separating slab gel is positioned against the upper stops 20 of the transfer device 10 and clamped in place. The second dimension 18 is cast with an extended stacking gel zone 22 as described above, or with a gap so that a stacking gel 22 may be cast in situ. In the case where the gel 22 is cast to the top of the slab 18 with the appropriate stacking gel, a flowable contacting medium (not shown), as described above, is placed at the opening of the gel slab 22. In the case of a cassette that is cast with a gap at the top, a liquid stacking gel mixture is placed in the gap to gel in situ. This is preferably applied so that it overflows the device, leaving no air bubbles or gaps, and may be modified through insertion of a comb, for application of additional samples.

The first dimension analytical gel 12 is placed in the positioning device so that it is in electrical communication with the opening of the second dimension slab gel 18 via the liquid, i.e., there is complete contact between the first dimension gel 12 and the second dimension gel 18 through either the flowable contacting medium or the stacking gel cast in situ. The transfer device is designed with a minimum of extraneous openings, so that during the second dimension electrophoretic separation the electric field passes substantially through the first dimension analytical gel, and not around it. The assembly of the first dimension analytical gel, transfer collar and second dimension slab is run in a manner known in the art in a suitable electrophoresis tank.

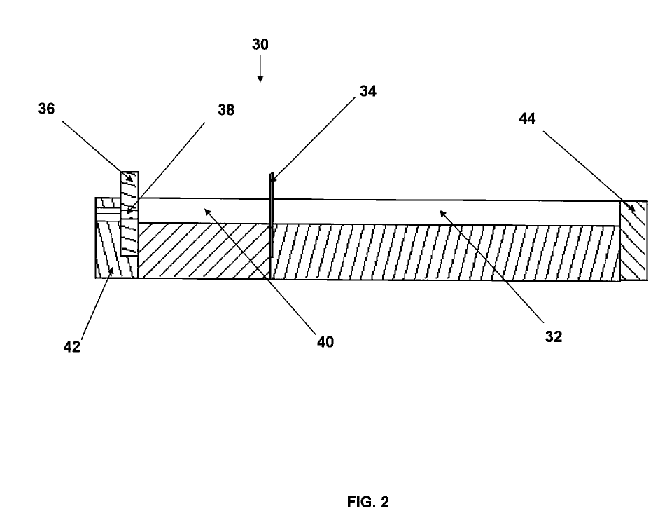

FIG. 2 depicts a horizontal format transfer device 30. First, the second dimension slab gel 32 is cast horizontally up to a partition 34. After the slab gel 32 is cast, the first dimension analytical gel 36, in this example, a dPC isoelectric focusing device, preferably equilibrated with SDS, is held firmly in place in a slot 38. With the dPC 36 in place and with the gel partition 34 removed, the stacking gel 40 is cast in the intermediate zone in such a way that there are no gaps between the first 36 and second dimension 32 gels. The second dimension electrophoresis is accomplished by contacting the first dimension with an appropriate cathode buffer via a slot in the cathode end cap 42, and with an appropriate anode buffer by removing the anode end cap 44.

The transfer device will typically be formed of a moldable or machinable thermoplastic such as a polycarbonate, polypropylene, or nylon, preferably non-conductive.

The construction and assembly and method of using the transfer device will be further understood by reference to the following non-limiting examples.

### EXAMPLE 1

### Flowable Transfer Gel

A hydroxypropyl methylcellulose flowable transfer gel for a second dimension molecular weight analysis was made in a standard Laemmli running buffer system. The Laemmli buffer composition was 62.5 mM TRIS-HCl, 25% (v/v) glycerol, 2% (w/v) SDS, and balance water. To be able to track the ion front during electrophoresis, 0.01% (w/v) bromophenol blue dye was added to the Laemmli buffer. The flowable transfer gel (30 ml) was made by first dry blending 0.90 g Methocel® K15M (Dow Chemical, Inc.) and 0.15 g Methocel® A5M. Then 30 ml of the Laemmli buffer dye mixture was heated to about 90° C. was placed in a 50 ml disposable centrifuge tube, to which the dry blend was added while agitating vigorously over a vortex mixer. The tube was then sealed an the mixture was allowed to hydrate overnight on a rocker table at room temperature. Entrapped bubbles were removed from the flowable gel by centrifuging the tube at about 3,000×g for 5 minutes.

For dispensing, a portion of the gel was transferred to a 10 ml syringe. Air bubbles were removed from the syringe by centrifugation at 3,000×g for 5 minutes.

### EXAMPLE 2

### Second Dimension Transfer

A first dimension dPC isoelectric trapping chip was transferred to a second dimension according to the following procedure. The chip was equilibrated for 10 minutes in an aqueous transfer buffer containing 3 M urea, 2% (w/v) SDS, 50 mM TRIS-HCl, and 0.01% (w/v) bromophenol blue.

The transfer collar, as depicted in FIG. 1, was affixed over the end of a conventional glass cassette slab gel that was cast with a polyacrylamide gel to its upper edge. Then a bead of the flowable transfer gel was deposited on the full length of the open end of the polyacrylamide gel, on top of which the dPC was pressed into the gel to its final special position as is determined by locating pins in the transfer collar. The assembly of transfer collar, dPC, flowable transfer gel and second dimension running gel was placed and run in a conventional electrophoresis device for running vertical glass second dimension cassettes.

Modifications and variations of the present invention will be apparent from the foregoing detailed description and accompanying figures. Such modifications and variations are intended to come within the scope of the following claims.

We claim:

1. A transfer device for improving uniformity and transfer efficiency in gel electropheresis of sample from a first dimensional gel to a second dimensional separation device positioned in an electropheresis chamber comprising anode and cathode chambers containing buffer, the device comprising
A collar for positioning samples in a first dimensional gel run in a first dimension in close abutment to a second dimensional separation device to be run in a second dimension relative to the cathode and anode chambers, comprising
Two ends and a top connecting the two ends, wherein the ends extend equidistant to form a space for insertion of a stacking buffer at the bottom of the transfer device,
Sealing means preventing leakage of cathode chamber buffer, and
Positioning means to clamp the collar onto the second dimensional separation device wherein the bottom of the transfer device is in abutment with the second dimensional separation device.

2. The device of claim 1 for positioning the first dimension with a second dimensional separation device selected from the group consisting of a molecular weight separation electropheresis gel, capillary electropheresis, liquid chromatography, and mass spectroscopy device.

3. The device of claim 1 comprising
Means for contacting the samples in the transfer collar with the second dimensional separation device, in the substantial absence of bubbles between the first dimension gel and the second dimensional separation device.

4. The device of claim 1 formed of polycarbonate non-conductive polymer.

5. The device of claim 1 wherein the transfer device retains the first dimensional gel in close abutment with the second dimensional separation device by means of at least one screw or clamp.

6. The device of claim 1 wherein the transfer collar comprises a recessed area on the bottom of the transfer device for positioning of the second dimensional separation device in close proximity to the stacking gel.

7. An electropheresis apparatus for electropheresis of sample from a first dimensional gel to a second dimensional gel comprising
A cathode chamber and cathode,
An anode chamber and anode,
Means for inserting and positioning a second dimensional gel, and
A transfer device for positioning samples in a first dimensional gel run in a first dimension in close abutment to a second dimensional separation device to be run in a second dimension relative to the cathode and anode chambers, the transfer device comprising a transfer collar and sealing means preventing leakage of cathode chamber buffer, and positioning means to clamp the transfer collar onto the second dimensional separation device.

8. The apparatus of claim 7 wherein the second dimensional device is selected from the group consisting of a molecular weight separation electropheresis gel, capillary electropheresis, liquid chromatography, and mass spectroscopy device.

9. The apparatus of claim 7 comprising at least one screw or clamp for securing the transfer collar in close proximity to the second dimensional separation device.

10. The apparatus of claim 7 wherein the apparatus comprises a slot adjacent to the cathode chamber for placement of the transfer device.

11. The apparatus of claim 7 wherein the apparatus comprises holes or slots to secure the screws or clamps on the transfer device to the second dimensional separation device.

12. A method for improving resolution of sample separated by application to a first dimensional gel when transferred to a second dimensional separation device comprising
Providing an electropheresis apparatus for electropheresis of sample from a first dimensional gel to a second dimensional gel comprising
A cathode chamber and cathode,
An anode chamber and anode, and
Means for inserting and positioning a second dimensional separating device, and
Providing a transfer device for positioning samples in a first dimensional gel run in a first dimension in close abutment to a second dimensional separation device to be run in a second dimension relative to the cathode and anode chambers, the transfer device comprising sealing means preventing leakage of cathode chamber buffer, and positioning means to clamp the collar onto the second dimensional separation device,
Positioning the second dimensional separation device within the apparatus,
Securing the transfer device to the second dimensional separation device, and
Filling the transfer collar with coupling medium or a stacking gel.

13. The method of claim 12 wherein the second dimensional separation device is selected from the group consisting of a molecular weight separation electropheresis gel, capillary electropheresis, liquid chromatography, and mass spectroscopy device.

14. The method of claim 12 wherein the first dimensional gel is an isoelectricfocusing gel and the second dimensional gel is a polyacrylamide gel for separation of molecules based on molecular weight.

15. The method of claim 12 wherein the transfer collar is overfilled with a stacking buffer.

16. The method of claim 12 wherein the transfer collar is filled with a liquid coupling medium.

17. The method of claim 12 wherein the second dimensional separation device is positioned in a vertical position.

18. The method of claim 17 wherein the second dimensional separation device is positioned and secured using screws or clamps through the transfer device into the apparatus holding the second dimensional separation device.

19. The method of claim 12 wherein the second dimensional separation device is positioned in a horizontal position.

20. The method of claim 12 wherein the transfer device is placed in close abutment to the second dimensional separation device in a slot adjacent to the cathode chamber.
